(19) United States
(12) Patent Application Publication
(10) Pub. No.: US 2024/0200007 A1
(43) Pub. Date: Jun. 20, 2024

(54) COMBINATORIAL FLUID SWITCH FOR USE IN AUTOMATED CELL ENGINEERING SYSTEMS

(71) Applicant: OCTANE BIOTECH INC., Kingston, Ontario (CA)

(72) Inventors: Peter SZARAZ, Kingston, Ontario (CA); Nuala TRAINOR, Kingston, Ontario (CA)

(21) Appl. No.: 18/554,768

(22) PCT Filed: Apr. 11, 2022
(86) PCT No.: PCT/US2022/024227

§ 371 Date: Oct. 10, 2023

Related U.S. Application Data
(60) Provisional application No. 63/173,719, filed on Apr. 12, 2021.

(51) Int. Cl. C12M 1/00; C12M 1/36

(52) U.S. Cl. CPC C12M 23/40; C12M 41/48

(57) ABSTRACT

The present disclosure provides combinatorial fluid switches that allow for the selection of a single fluid flow-path, while controlling multiple flow-paths. The combinatorial fluid switches can be used in various biological systems and processes, included automated cell engineering systems.

### CROSS-RELATED APPLICATIONS

The present application claims the benefit of U.S. Provisional Appl. No. 63/173,719, entitled “COMBINATORIAL FLUID SWITCH FOR USE IN AUTOMATED CELL ENGINEERING SYSTEMS” and filed Apr. 12, 2021, the entire content of which is incorporated by reference herein.

### FIELD OF INVENTION

The present disclosure provides combinatorial fluid switches that allow for the selection of a single fluid flow-path, while controlling flow through multiple flow-paths. The combinatorial fluid switches can be used in various biological systems and processes, included automated cell engineering systems.

### BACKGROUND OF THE INVENTION

As anticipation builds about accelerated clinical adoption of advanced cell therapies, more attention is turning to the underlying manufacturing strategies that will allow these therapies to benefit patients worldwide. While cell therapies hold great promise clinically, high manufacturing costs relative to reimbursement present a formidable roadblock to commercialization. Thus, the need for cost effectiveness, process efficiency and product consistency is driving efforts for automation in numerous cell therapy fields.

Automation of various processes is involved in producing cell populations for therapy. This includes integration of cell activation, transduction and expansion into a commercial manufacturing platform, for the translation of these important therapies to the broad patient population.

In such automated cell engineering systems, it is often necessary to control the flow of one or more fluids through various valve constructs to deliver cells, media, vector solutions, etc., to desired areas or chambers of a system. The present invention provides the design of a combinatorial fluid switch to control this fluid flow using a reduced number of valves.

### SUMMARY OF THE INVENTION

In some embodiments, provided herein is a combinatorial fluid switch for controlling fluid flow through a plurality of fluid flow-paths, comprising: a plurality of fluid inputs; a first two-position valve having individual fluid flow-paths, through which fluids from the plurality of fluid inputs are guided; a second two-position valve having individual fluid flow-paths, through which fluids from the plurality of fluid inputs are guided; and a plurality of fluid outputs, wherein the combinatorial fluid switch is configured to allow: fluid flow from a first fluid input to a first fluid output when the first two-position valve is in an open position and the second two-position valve is in an open position; fluid flow from a second fluid input to a second fluid output when the first two-position valve is in a closed position and the second two-position valve is in an open position; fluid flow from a third fluid input to a third fluid output when the first two-position valve is in an open position and the second two-position valve is in a closed position; and fluid flow from a fourth fluid input to a fourth fluid output when the first two-position valve is in a closed position and the second two-position valve is in a closed position.

In further embodiments, provided herein is a combinatorial fluid switch for controlling fluid flow through at least four fluid flow-paths, comprising: a first, a second, a third and a fourth fluid inputs; a first two-position valve having four fluid flow-paths, through which fluids from the first, second, third and fourth fluid inputs are guided; a second two-position valve having four fluid flow-paths, through which fluids from the first, second, third and fourth fluid inputs are guided; and a first, a second, a third and a fourth fluid outputs, wherein the combinatorial fluid switch is configured to allow: fluid flow from the first fluid input to the first fluid output when the first two-position valve is in an open position and the second two-position valve is in an open position; fluid flow from the second fluid input to the second fluid output when the first two-position valve is in a closed position and the second two-position valve is in an open position; fluid flow from the third fluid input to the third fluid output when the first two-position valve is in an open position and the second two-position valve is in a closed position; and fluid flow from the fourth fluid input to the fourth fluid output when the first two-position valve is in a closed position and the second two-position valve is in a closed position.

In still further embodiments, provided herein is a system for controlling fluid flow through at least sixteen fluid flow-paths, comprising: a first combinatorial fluid switch comprising: a first, a second, a third and a fourth fluid inputs; a first two-position valve having four fluid flow-paths, through which fluids from the first, second, third and fourth fluid inputs are guided; a second two-position valve having four fluid flow-paths, through which fluids from the first, second, third and fourth fluid inputs are guided; and a first, a second, a third and a fourth fluid outputs, wherein the first combinatorial fluid switch is configured to allow: fluid flow from the first fluid input to the first fluid output when the first two-position valve is in an open position and the second two-position valve is in an open position; fluid flow from the second fluid input to the second fluid output when the first two-position valve is in a closed position and the second two-position valve is in an open position; fluid flow from the third fluid input to the third fluid output when the first two-position valve is in an open position and the second two-position valve is in a closed position; and fluid flow from the fourth fluid input to the fourth fluid output when the first two-position valve is in a closed position and the second two-position valve is in a closed position; and a second combinatorial fluid switch fluidly connected to the first combinatorial fluid switch, the second combinatorial fluid switch comprising: a fifth, a sixth, a seventh and an eighth fluid inputs; a third two-position valve having four fluid flow-paths, through which fluids from the fifth, sixth, seventh and eighth fluid inputs are guided; a fourth two-position valve having four fluid flow-paths, through which fluids from the fifth, sixth, seventh and eighth fluid inputs are guided; and a fifth, a sixth, a seventh and an eighth fluid outputs, wherein the second combinatorial fluid switch configured to allow: fluid flow from the fifth fluid input to the fifth fluid output when the third two-position valve is in an open position and the fourth two-position valve is in an open position; fluid flow from the sixth fluid input to the sixth fluid output when the third two-position valve is in a closed position and the third two-position valve is in an open position; fluid flow from the seventh fluid input to the seventh fluid output when the third two-position valve is in an open position and the fourth two-position valve is in a closed position; and fluid flow from the eighth fluid input to the eighth fluid output when the third two-position valve is in a closed position and the fourth two-position valve is in a closed position.

In still other embodiments, provided herein is a system for controlling fluid flow through at least sixteen fluid flow-paths, comprising: a plurality of combinatorial fluid switches, each combinatorial fluid switch comprising: a first, a second, a third and a fourth fluid inputs; a first two-position valve having four fluid flow-paths, through which fluids from the first, second, third and fourth fluid inputs are guided; a second two-position valve having four fluid flow-paths, through which fluids from the first, second, third and fourth fluid inputs are guided; and a first, a second, a third and a fourth fluid outputs, wherein the first combinatorial fluid switch is configured to allow: fluid flow from the first fluid input to the first fluid output when the first two-position valve is in an open position and the second two-position valve is in an open position; fluid flow from the second fluid input to the second fluid output when the first two-position valve is in a closed position and the second two-position valve is in an open position; fluid flow from the third fluid input to the third fluid output when the first two-position valve is in an open position and the second two-position valve is in a closed position; and fluid flow from the fourth fluid input to the fourth fluid output when the first two-position valve is in a closed position and the second two-position valve is in a closed position.

In still further embodiments, provided herein is an automated biologic production system, comprising: an enclosable housing; a cassette contained within the enclosable housing, the cassette comprising: a cell culture chamber a combinatorial fluid switch as described herein; a pumping system fluidly connected to the cell culture chamber and the combinatorial switch; one or more of a temperature sensor, a pH sensor, a glucose sensor, a lactose sensor, an oxygen sensor, a carbon dioxide sensor, and an optical density sensor; and mechanisms to automatically adjust one or more of a temperature, a pH level, a glucose level, a lactose level, an oxygen level, a carbon dioxide level, and an optical density.

In additional embodiments, provided herein is an automated biologic production system, comprising: an enclosable housing; a cassette contained within the enclosable housing, the cassette comprising: a cell culture chamber; a system as described herein; a pumping system fluidly connected to the cell culture chamber and the system; one or more of a temperature sensor, a pH sensor, a glucose sensor, a lactose sensor, an oxygen sensor, a carbon dioxide sensor, and an optical density sensor; and mechanisms to automatically adjust one or more of a temperature, a pH level, a glucose level, a lactose level, an oxygen level, a carbon dioxide level, and an optical density.

Also provided herein is a combinatorial fluid switch, comprising: a housing having two opposing sides, each side having four openings passing therethrough; and two, two-position valves disposed within the housing, each valve having four openings passing therethrough, wherein the openings in the sides and the openings in the two-position valves are configured to receive tubing therethrough, so as to create four flow-paths within the combinatorial fluid switch, and wherein the two-position valves are moveable within the housing to allow fluid flow through only one flow-path at a time.

In further embodiments, provided herein is a combinatorial fluid switch, comprising: a support base comprising two raised portions and two recessed portions, the two raised portions comprising a plurality of partitions extending above the support base configured to allow tubing to pass therethrough so as to create four fluid flow-paths, the two recessed portions each comprising four stationary compression members extending above the support base; two, two-position valves having openings to allow the stationary compression members to pass through, the valves further comprising four movable compression members configured to compress against a complementary, stationary compression member on the support base so as to constrict a tubing between the movable compression member and the complementary, stationary compression member, wherein the two-position valves are configured to slide along the support base and allow fluid flow through only one flow-path at a time.

In still further embodiments, provided herein is a combinatorial fluid switch, comprising: at least three, stationary compression members; a first two-position valve having two movable compression members integrated into the valve; and a second two-position valve having three movable compression members integrated into the valve, wherein the movable compression members of the first and second two-position valves are configured to compress against a complementary, stationary compression member so as to constrict a tubing between the movable compression member and the complementary, stational compression member, wherein the two-position valves are configured to slide and allow fluid flow through one flow-path at a time.

In additional embodiments, provided herein is a combinatorial fluid switch for controlling fluid flow through a plurality of fluid flow-paths, comprising: a plurality of fluid inputs; a first control valve, through which fluids from the plurality of fluid inputs are guided; a second control valve having individual fluid flow-paths, through which fluids from the plurality of fluid inputs are guided; and a plurality of fluid outputs, wherein the combinatorial fluid switch is configured to allow fluid flow through a designated combination of fluid inputs and fluid outputs.

In further embodiments, provided herein is a method for controlling fluid flow within a closed, cell engineering system, comprising: providing a plurality of fluid inputs; providing a plurality of fluid outputs; providing a plurality of flow-paths connecting the fluid inputs to the fluid outputs; providing a plurality of valves controlling the flow within the flow-paths, thereby controlling the direction, speed, duration and/or interval of fluid flow within the flow-paths.

### BRIEF DESCRIPTION OF THE FIGURES

FIGS. 1A-1D show combinatorial fluid switches in accordance with embodiments hereof.

FIGS. 2A-2D show exemplary combinatorial fluid switches.

FIGS. 3A-3B show systems for controlling fluid flow in accordance with embodiments hereof.

FIG. 4A shows a representation of a cassette that includes combinatorial fluid switches in accordance with embodiments hereof.

FIGS. 4B-4C show automated biologic production systems in accordance with embodiments hereof.

FIG. 5 shows an example of a combinatorial fluid switch in accordance with embodiments hereof.

FIG. 6 shows a further example of a combinatorial fluid switch in accordance with embodiments hereof.

FIGS. 7A-7C show an additional example of a combinatorial fluid switch in accordance with embodiments hereof.

FIGS. 8A-8C show the integration of combinatorial fluid switches into an automated cell engineering system in accordance with embodiments hereof.

FIG. 9 shows the connection of systems as described herein.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

In embodiments, provided herein is a combinatorial fluid switch for controlling fluid flow through a plurality of fluid flow-paths. As used herein a “fluid switch” refers to a combination of at least one valve and at least one fluid flow-path, where the valve controls fluid flow through the flow-path, and in embodiments, either allows fluid flow (“on”), or completely stops fluid flow (“off”). A “combinatorial fluid switch” refers to a combination of a plurality of valves that, in various combinations of open and closed positions, control the flow of fluid through a plurality of fluid flow-paths. The concept of a “combinatorial” switch comes from the fact that placing multiple valves together allows for the control of multiple flow-paths.

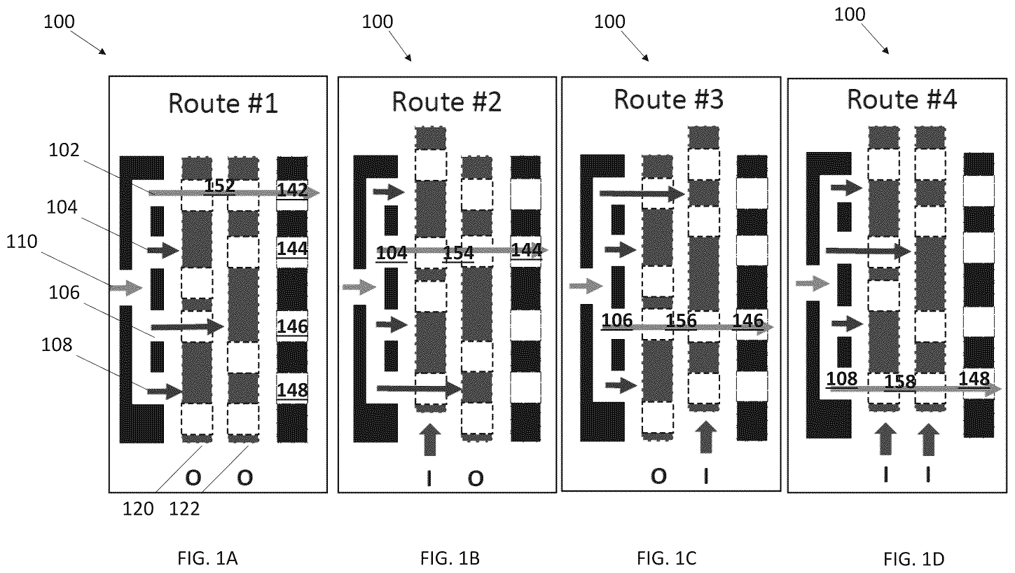

FIGS. 1A-1D show exemplary combinatorial fluid switches, in which the switches comprise a plurality of fluid inputs, a first two-position valve having individual fluid flow-paths, through which fluids from the plurality of fluid inputs are guided, a second two-position valve having individual fluid flow-paths, through which fluids from the plurality of fluid inputs are guided, and a plurality of fluid outputs. As described herein the combinatorial fluid switch is configured to allow fluid flow from a first fluid input to a first fluid output when the first two-position valve is in an open position and the second two-position valve is in an open position, fluid flow from a second fluid input to a second fluid output when the first two-position valve is in a closed position and the second two-position valve is in an open position, fluid flow from a third fluid input to a third fluid output when the first two-position valve is in an open position and the second two-position valve is in a closed position, and fluid flow from a fourth fluid input to a fourth fluid output when the first two-position valve is in a closed position and the second two-position valve is in a closed position.

In contrast to the combinatorial fluid switches described herein, with individual valve systems, each has 2 positions, open and closed, and individually 1 actuator, 1 valve, and 1 tube. However, the theoretical maximum number of combinations that can be achieved by valves or on/off switches is 2ⁿ, where n is the number of valves included. Thus, for two valves, the number of combinations that can be achieved is 4.

Thus, as shown in FIGS. 1A-1D, a combinatorial fluid switch 100 can be constructed using 2 switches, that allows for the control of four fluid flow-paths. Thus, in embodiments, provided herein is a combinatorial fluid switch for controlling fluid flow through at least four fluid flow-paths. As used herein a “fluid flow-path” refers to a conduit, channel, tube, tunnel, or similar structure that allows for fluid (suitably a liquid) to pass through from an inlet to the flow-path through the flow-path, to an outlet of the flow-path.

As shown in FIG. 1A, combinatorial fluid switch 100 suitably includes a first fluid input 102, a second fluid input 104, a third fluid input 106 and a fourth fluid input 108. The combinatorial switch also suitably includes a first two-position valve 120, having four fluid flow-paths (152, 154, 156 and 158), through which fluids from the first 102, second 104, third 106 and fourth 108 inputs are guided. The combinatorial switch also suitably includes a second, two-position valve 122, through which fluids from the first 102, second 104, third 106 and fourth 108 inputs are guided. As shown in FIG. 1A, combinatorial switch 100 also includes a first fluid output 142, a second fluid output 144, a third fluid output 146 and a fourth fluid output 148.

As shown illustratively in FIGS. 1A-1D, combinatorial switch 100 is configured to allow (when valves 120 and 122 are open “0” or closed “1”):
fluid flow (via flow-path 152) from the first fluid input 102 to the first fluid output 142 when the first two-position valve is in an open position and the second two-position valve is in an open position;
fluid flow (via flow-path 154) from the second fluid input 104 to the second fluid output 144 when the first two-position valve is in a closed position and the second two-position valve is in an open position;
fluid flow (via flow-path 156) from the third fluid input 106 to the third fluid output 106 when the first two-position valve is in an open position and the second two-position valve is in a closed position; and
fluid flow (via flow-path 158) from the fourth fluid input 108 to the fourth fluid output 148 when the first two-position valve is in a closed position and the second two-position valve is in a closed position.

For example, as shown in FIG. 1B, when first two-position valve 120 is closed (“1”), flow-path 152 is blocked (see top of FIG. 1B) as is flow-path 156 (see middle of FIG. 1B), by first two-position valve 120, while flow-path 158 is blocked by second two-position valve 122 (see bottom of FIG. 1B). However, flow-path 154 is open and thus, fluid can flow from input 104 through to output 144, via flow-path 154.

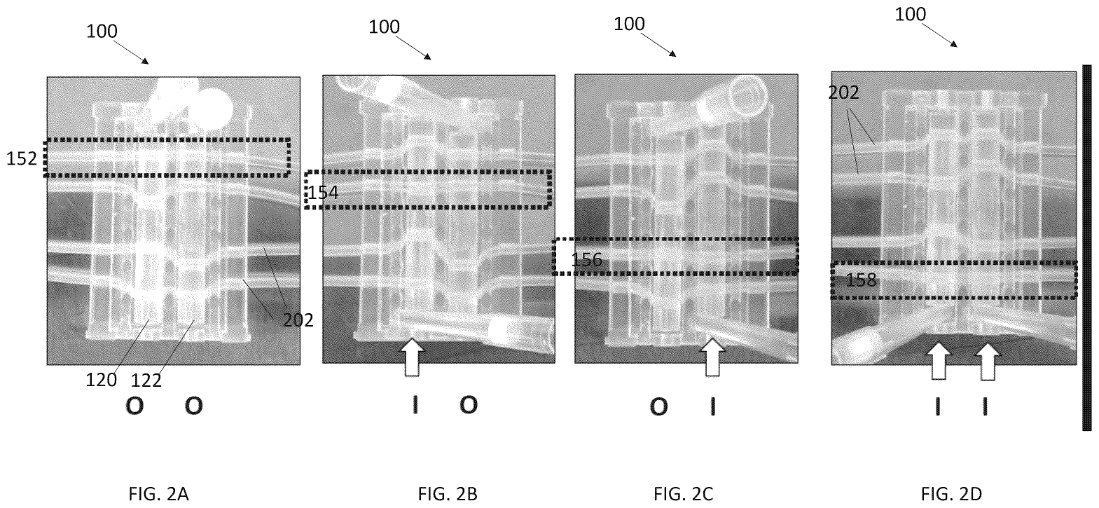

FIGS. 2A-2D, show an exemplary combinatorial switch 100 in which each of the fluid inputs comprising tubing, and the fluid flow-paths (152, 154, 156, 158) also suitably comprise tubing. As illustrated in FIGS. 2A-2D, in embodiments, the two-position valves 120 and 122 are trumpet valves—i.e., valves that have two positions (open “0” or closed “1”) and are capable of compressing or pinching flow-paths (e.g., tubing. Suitably, as shown in FIGS. 2A-2D, in embodiments, the fluid inputs, the fluid flow-paths and the fluid outputs, consist of at least four tubing lines. In suitable embodiments, the trumpet valves include a shield or covering (suitably a silicone covering or similar material) that limits contamination and maintains the sterility of the trumpet valves and thus the flow paths where they are utilized.

Exemplary materials for use in tubing for the flow-paths, inputs and outputs, are known in the art and include for example, various polymers (e.g., silicone tubing), plastics, glass, metal, ceramics, composites, etc.

In additional embodiments, each of the fluid inputs (102, 104, 106 and 108) can be fed from a single fluid source 110 (see e.g., FIG. 1A). Suitably, the fluids within the fluid flow-paths are not allowed to mix with each other. This can be accomplished through the use of tubing components for each of the fluid flow-paths, or distinct conduits or other passages that do not interact with each other.

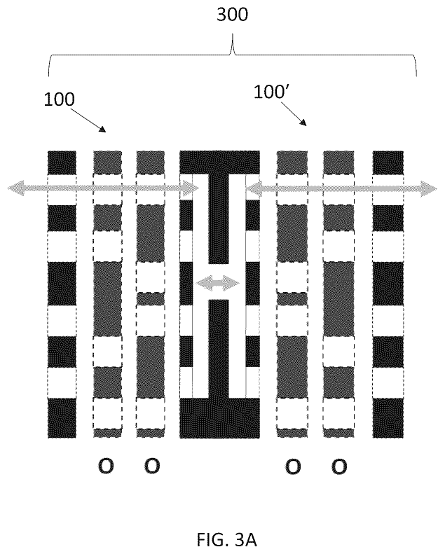

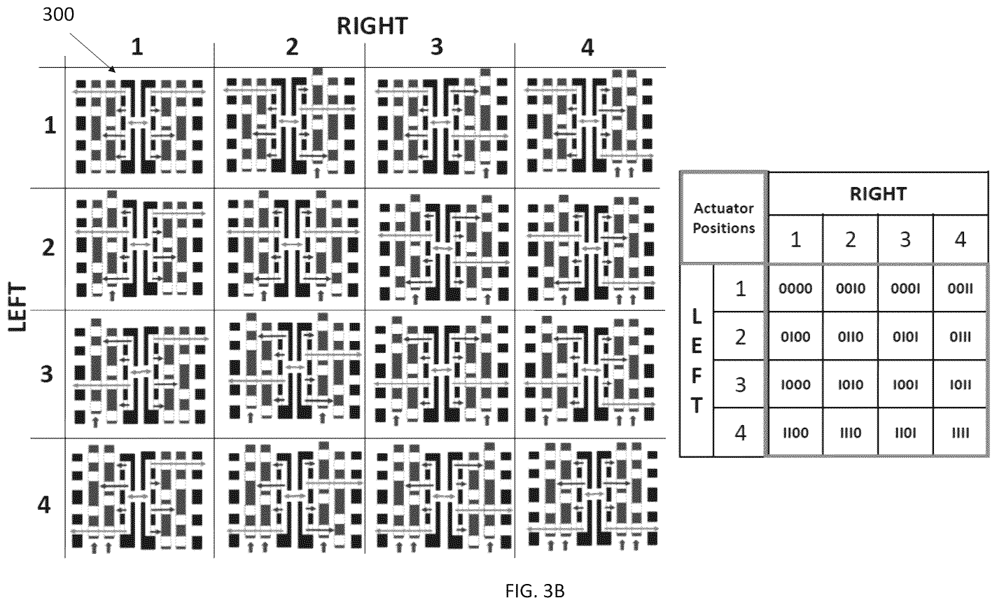

In further embodiments, provided herein is a system for controlling fluid flow. In exemplary embodiments, the system controls fluid flow through at least sixteen fluid flow-paths. As shown in FIG. 3A, system 300 suitably comprises a first combinatorial fluid switch 100, as described herein, and for example a first, a second, a third and a fourth fluid inputs, a first two-position valve having four fluid flow-paths, through which fluids from the first, second, third and fourth fluid inputs are guided, a second two-position valve having four fluid flow-paths, through which fluids from the first, second, third and fourth fluid inputs are guided, and a first, a second, a third and a fourth fluid outputs. As described herein, the first combinatorial fluid switch is configured to allow: fluid flow from the first fluid input to the first fluid output when the first two-position valve is in an open position and the second two-position valve is in an open position; fluid flow from the second fluid input to the second fluid output when the first two-position valve is in a closed position and the second two-position valve is in an open position; fluid flow from the third fluid input to the third fluid output when the first two-position valve is in an open position and the second two-position valve is in a closed position; and fluid flow from the fourth fluid input to the fourth fluid output when the first two-position valve is in a closed position and the second two-position valve is in a closed position.

System 300 also further includes a second combinatorial fluid switch 100′ fluidly connected to the first combinatorial fluid switch (see fluid connection between first combinatorial switch 100 and second combinatorial switch 100′ in FIG. 3A). Suitably, second combinatorial fluid switch 100′ comprising a fifth, a sixth, a seventh and an eighth fluid inputs, a third two-position valve having four fluid flow-paths, through which fluids from the fifth, sixth, seventh and eighth fluid inputs are guided, a fourth two-position valve having four fluid flow-paths, through which fluids from the fifth, sixth, seventh and eighth fluid inputs are guided; and a fifth, a sixth, a seventh and an eighth fluid outputs. As described herein, second combinatorial fluid switch 100′ is configured to allow: fluid flow from the fifth fluid input to the fifth fluid output when the third two-position valve is in an open position and the fourth two-position valve is in an open position; fluid flow from the sixth fluid input to the sixth fluid output when the third two-position valve is in a closed position and the third two-position valve is in an open position; fluid flow from the seventh fluid input to the seventh fluid output when the third two-position valve is in an open position and the fourth two-position valve is in a closed position; and fluid flow from the eighth fluid input to the eighth fluid output when the third two-position valve is in a closed position and the fourth two-position valve is in a closed position.

It should be understood that the number of fluid inputs, flow paths, and fluid outputs, is utilized simply to illustrate the combinatorial nature of the switches described herein. Numerical numbering does not imply that the valves must be utilized in any specific order or orientation (i.e., valves can be selected in any order and in any sequence).

In still further embodiments, system 300 for controlling fluid flow through at least sixteen fluid flow-paths, can include a plurality of combinatorial fluid switches (e.g., 100 and 100′), each combinatorial fluid switch comprising: a first, a second, a third and a fourth fluid inputs; a first two-position valve having four fluid flow-paths, through which fluids from the first, second, third and fourth fluid inputs are guided; a second two-position valve having four fluid flow-paths, through which fluids from the first, second, third and fourth fluid inputs are guided; and a first, a second, a third and a fourth fluid outputs, wherein the first combinatorial fluid switch is configured to allow: fluid flow from the first fluid input to the first fluid output when the first two-position valve is in an open position and the second two-position valve is in an open position; fluid flow from the second fluid input to the second fluid output when the first two-position valve is in a closed position and the second two-position valve is in an open position; fluid flow from the third fluid input to the third fluid output when the first two-position valve is in an open position and the second two-position valve is in a closed position; and fluid flow from the fourth fluid input to the fourth fluid output when the first two-position valve is in a closed position and the second two-position valve is in a closed position.

In suitable embodiments, each of the combinatorial fluid switches (e.g., 100 and 100′) is fluidly connected to each other. As used herein “fluidly connected” refers to a junction or meeting between two fluid switches that allows for fluid to pass form one switch to the other without loss of volume, and without mixing between fluids (unless mixing is desired).

In embodiments, system 300 can include 4 or more combinatorial fluid switches, such as 5 or more, 6 or more, 7 or more, 8 or more, 9 or more, 10 or more, 11 or more, 12 or more, 13 or more, 14 or more, 15 or more, 16 or more, 17 or more, 18 or more, 19 or more or 20 or more fluid switches, etc.

As described throughout, suitably the fluid inputs, fluid flow-paths, and fluid outputs comprise tubing, and the valves are trumpet valves. In exemplary embodiments, the fluid inputs, the fluid flow-paths and the fluid outputs consist of at least eight tubing lines.

FIG. 3B shows the sixteen (16) different flow-paths that can be achieved via the use of system 300, if there are four inputs, four flow paths, and four outputs, per valve pairs. As shown in FIG. 3B, each of the two position valves (four in total) have an open position (0) and a closed position (1). By selected the two available positions for each of the four valves in all of the possible combinations (16 total), sixteen (16) different flow-paths are selected. The matrix in the lower right of FIG. 3B shows the 16 valve position combinations, and the flow-path matrix shows the 16 different flow-paths.

As illustrated, with the use of just 4, two-position valves, 16 total flow-paths are possible (24). This illustrates the significant advantage of the systems 300 that comprise fluidly connected combinatorial switches 100, dramatically expanding the number of flow-paths that can be achieved, relative to valves positioned in a traditional orientation.

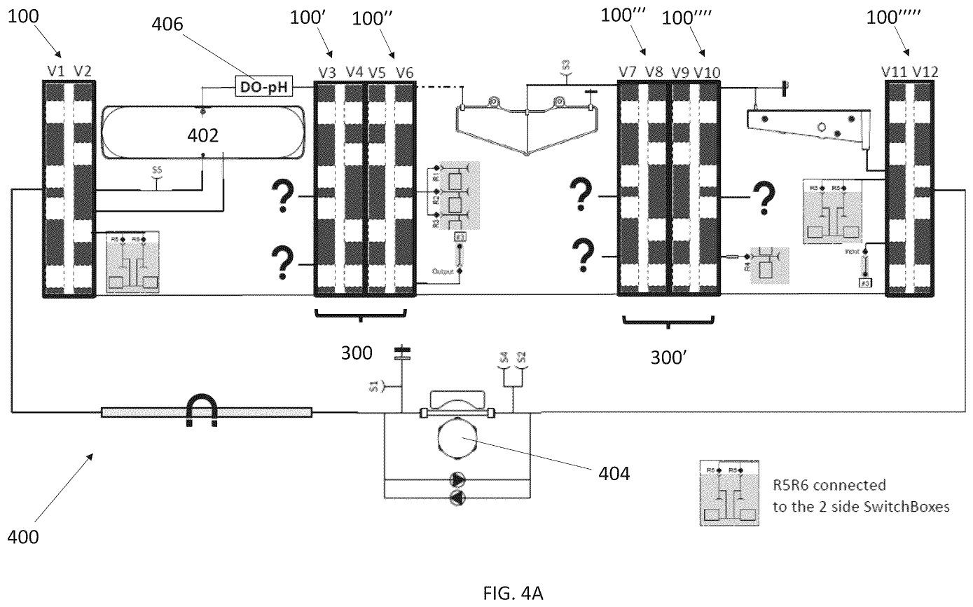

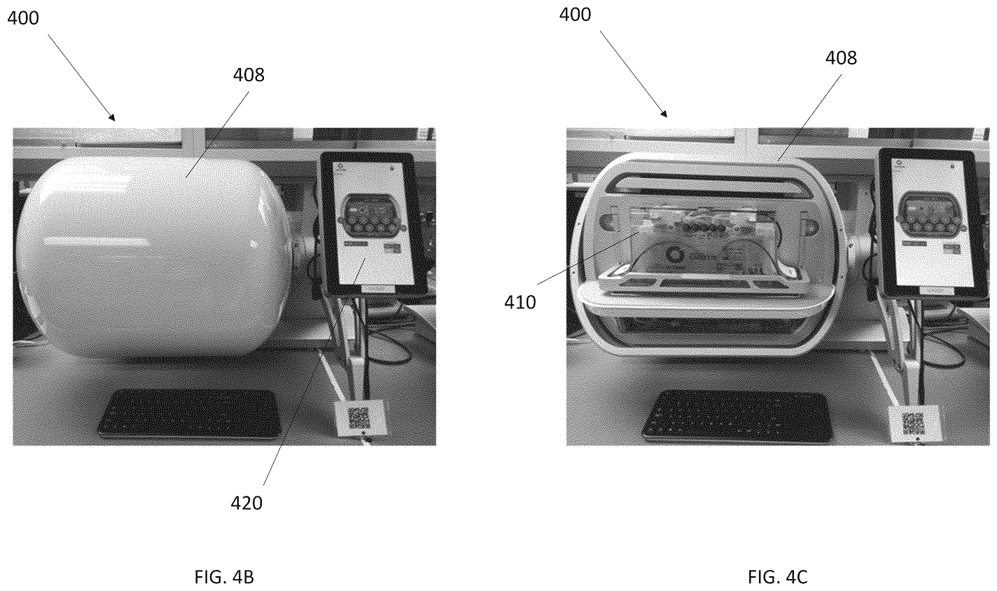

In still further embodiments, provided herein are automated biologic production systems that include the combinatorial fluid switches described herein. FIG. 4A shows a schematic of an automated biologic production system 400, as described herein, and FIGS. 4B and 4C show examples of automate biologic production systems 400 that include cassettes for the production of biologic products, including antibodies, proteins, cells, etc.

As described herein, the cassettes and methods are utilized and carried out in a fully enclosed automated biologic production systems, e.g. an automated cell engineering system 400 (see FIGS. 4B and 4C), suitably for performing steps such as, activating, transducing, expanding, concentrating, and harvesting. Cell engineering systems for automated production of, for example genetically modified immune cells, including CAR T cells, are described in U.S. Published Patent Application No. 2019/0169572 (the disclosure of which is incorporated by reference herein in its entirety), and are also called automated cell engineering system, COCOON™, or COCOON™ system herein.

For example, a user can provide an automated cell engineering system pre-filled with a cell culture and reagents (e.g., an activation reagent, a vector, cell culture media, nutrients, selection reagent, and the like) and parameters for the cell production (e.g., starting number of cells, type of media, type of activation reagent, type of vector, number of cells or doses to be produced, and the like). The automated cell engineering system is able to carry out various automated methods, including methods of producing genetically modified immune cell cultures, including CAR T cells, without further input from the user. In some embodiments, the fully enclosed automated cell engineering system minimizes contamination of the cell cultures by reducing exposure of the cell culture to non-sterile environments. In additional embodiments, the fully enclosed automated cell engineering system minimizes contamination of the cell cultures by reducing user handling of the cells.

In embodiments, the automated biologic production system (cell engineering system) 400 (see FIGS. 4A-4C) includes an enclosable housing 408, a cassette 410 contained within the enclosable housing 408, the cassette comprising: a cell culture chamber 402, a combinatorial fluid switch 100 as described herein, a pumping system 404 fluidly connected to the cell culture chamber 402 and the combinatorial switch 100. The automated biologic production system 400 also further includes one or more of a temperature sensor, a pH sensor (e.g., 406), a glucose sensor, a lactose sensor, an oxygen sensor, a carbon dioxide sensor, and an optical density sensor; and mechanisms to automatically adjust one or more of a temperature, a pH level, a glucose level, a lactose level, an oxygen level, a carbon dioxide level, and an optical density. As shown in FIG. 4A, combinatorial switches can be positioned in the flow-path of the automated biologic production system 400 at various positions (see 100 and 100′″″).

As used herein, “enclosable housing” refers to a structure than can be opened and closed, and within which cassette 410 as described herein, can be placed and integrated with various components such as fluid supply lines, gas supply lines, power, cooling connections, heating connections, etc. As shown in FIGS. 4B and 4C, enclosable housing can be opened (FIG. 4C) to allow insertion of the cassette, and closed (FIG. 4B) to maintain a closed, sealed environment to allow the various automated processes described herein to take place utilizing the cassette.

In other embodiments, the automated biologic production systems 400 can include one or more systems 300 and 300′, as shown in FIG. 4A. In such embodiments, automated biologic production system 400 suitably includes enclosable housing 408, a cassette 410 contained within the enclosable housing, the cassette comprising: a cell culture chamber 401, a system 300 as described herein, a pumping system 404 fluidly connected to the cell culture chamber and the system 300 one or more of a temperature sensor, a pH sensor (e.g., 406), a glucose sensor, a lactose sensor, an oxygen sensor, a carbon dioxide sensor, and an optical density sensor; and mechanisms to automatically adjust one or more of a temperature, a pH level, a glucose level, a lactose level, an oxygen level, a carbon dioxide level, and an optical density.

In exemplary embodiments, automated biologic production systems 400 can further include one or more of a magnetic cell separation device or an electroporation device. The automated biologic production systems 400 suitably include at least 16 fluid flow-paths, including for example, at least 17 fluid flow-paths, at least 18 fluid flow-paths, at least 19 fluid flow-paths, at least 20 fluid flow-paths, at least 21 fluid flow-paths, at least 22 fluid flow-paths, at least 23 fluid flow-paths, at least 24 fluid flow-paths, at least 25 fluid flow-paths, at least 26 fluid flow-paths, at least 27 fluid flow-paths, at least 28 fluid flow-paths, at least 29 fluid flow-paths, or at least 30 fluid flow-paths.

As described throughout, the automated biologic production systems 400 described herein are suitably configured to produce cells, e.g., CAR-T cells.

As described herein, the automated biologic production systems 400, including automated cell engineering systems suitably include a cassette 410. Thus, in embodiments, provided herein is a cassette for use in an automated cell engineering system that includes one or more combinatorial fluid switches 100, and/or systems 300 that include the combinatorial fluid switches 100, as described throughout. As used herein a “cassette” refers to a largely self-contained, removable and replaceable element of a automated biologic production (cell engineering) system that includes one or more chambers for carrying out the various elements of the methods described herein, and suitably also includes one or more of a cell media, an activation reagent, a wash media, etc.

Cassette 410 suitably includes a cellular sample input. Cellular sample input can be a vial or chamber in which a cellular sample can be placed prior to introduction or loading into cassette 410. In other embodiments, cellular sample input can simply be a sterile-locking tubing (for example a luer lock tubing connection or the like) to which a syringe or a cell-containing bag, such as a blood bag, can be connected.

Exemplary fluid connections that can be used in cassette 410 to connect the various components, including combinatorial fluid switches 100 and systems 300, include various tubing, channels and connections known in the art, such as silicone or rubber tubing, luer lock connections, etc. It should be understood that components that are fluidly connected can also include additional elements between each of the components, while still maintaining a fluid connection. That is, fluidly connected components can include additional elements, such that a fluid passing between the components can also pass through these additional elements, but is not required to do so.

Pumping system 404 is suitably a peristaltic pump system, though other pumping systems can also be utilized. A peristaltic pump refers to a type of positive displacement pump for pumping a fluid. The fluid is suitably contained within a flexible tube fitted inside a pump casing—often circular. A rotor with a number of “rollers”, “shoes”, “wipers”, or “lobes” attached to the external circumference of the rotor compresses the flexible tube. As the rotor turns, the part of the tube under compression is pinched closed (or “occludes”) thus forcing the fluid to be pumped to move through the tube. Additionally, as the tube opens after the passing of the cam (“restitution” or “resilience”) fluid flow is induced to the pump. This process is called peristalsis and is used to move fluid through the flexible tube. Typically, there are two or more rollers, or wipers, occluding the tube, trapping between them a body of fluid. The body of fluid is then transported toward the pump outlet.

As described herein, a magnetic separation process can be utilized to eliminate and separate undesired cells and debris from a cell population. In such embodiments, a magnetic bead or other structure, to which a biomolecule (e.g., antibody, antibody fragment, etc.) has been bound, can interact with a target cell. Various magnetic separation methods, including the use of filters, columns, flow tubes or channels with magnetic fields, etc., can then be used to separate the target cell population from undesired cells, debris, etc., that may be in a cellular sample. For example, a target cell population can flow through a tube or other structure and be exposed to a magnetic field, whereby the target cell population is retained or held-up by the magnetic field, allowing undesired cells and debris to pass through the tube. The magnetic field can then be turned off, allowing the target cell population to pass onto a further retention chamber or other area(s) of the cassette for further automated processing. Additional filtration includes traditional column filtration, or use of other filtration membranes and structures.

In exemplary embodiments, cell culture chamber 402 is a flat and non-flexible chamber (i.e., made of a substantially non-flexible material such as a plastic) that does not readily bend or flex. The use of a non-flexible chamber allows the cells to be maintained in a substantially undisturbed state. In embodiments, the overall thickness of cell culture chamber 402 (i.e., the chamber height) is low, on the order of about 0.5 cm to about 5 cm. Suitably, the cell culture chamber has a volume of between about 0.50 ml and about 500 ml, or about 1 ml to about 300 ml, more suitably between about 50 ml and about 200 ml, or the cell culture chamber has a volume of about 180 ml. The use of a low chamber height (less than 5 cm, suitably less than 4 cm, less than 3 cm, or less then 2 cm) allows for effective media and gas exchange in close proximity to the cells. Ports are configured to allow mixing via recirculation of the fluid without disturbing the cells. Larger height static vessels can produce concentration gradients, causing the area near the cells to be limited in oxygen and fresh nutrients. Through controlled flow dynamics, media exchanges can be performed without cell disturbance. Media can be removed from the additional chambers (no cells present) without risk of cell loss.

As described herein, in exemplary embodiments the cassette is pre-filled with one or more of a cell culture, a culture media, a cell wash media if desired, an activation reagent, and/or a vector, including any combination of these. In further embodiments, these various elements can be added later via suitable injection ports, etc.

As described herein, in embodiments, the cassettes suitably further include one or more of a pH sensor 406, a glucose sensor (not shown), an oxygen sensor, a carbon dioxide sensor (not shown), a lactic acid sensor/monitor (not shown), and/or an optical density sensor (not shown). The cassettes can also include one or more sampling ports and/or injection ports. Examples of such sampling ports and injection ports can include an access port for connecting the cartridge to an external device, such as an electroporation unit or an additional media source.

In embodiments, cassette 410 suitably includes a low temperature chamber, which can include a refrigeration area suitably for storage of a cell culture media, as well as a high temperature chamber, suitably for carrying out activation, transduction and/or expansion of a cell culture. Suitably, the high temperature chamber is separated from the low temperature chamber by a thermal barrier. As used herein “low temperature chamber” refers to a chamber, suitably maintained below room temperature, and more suitably from about 4° C. to about 8° C., for maintenance of cell media, etc., at a refrigerated temperature. As used herein “high temperature chamber” refers to chamber, suitably maintained above room temperature, and more suitably maintained at a temperature to allow for cell proliferation and growth, i.e., between about 35-40° C., and more suitably about 37° C. In embodiments, high temperature chamber suitably includes cell culture chamber 206 (also called proliferation chamber or cell proliferation chamber throughout).

Fluidics pathways, including gas exchange lines, may be made from a gas-permeable material such as, e.g., silicone. In some embodiments, the automated cell engineering system recirculates oxygen throughout the substantially non-yielding chamber during the cell production methods. Thus, in some embodiments, the oxygen level of a cell culture in the automated cell engineering system is higher than the oxygen level of a cell culture in a flexible, gas-permeable bag. Higher oxygen levels may be important in the cell culture expansion step, as increased oxygen levels may support increased cell growth and proliferation.

In embodiments, the methods and cartridges described herein are utilized in the COCOON® platform (Octane Biotech (Kingston, ON)), which integrates multiple unit operations in a single turnkey platform. Multiple cell protocols are provided with very specific cell processing objectives. To provide efficient and effective automation translation, the methods described utilize the concept of application-specific/sponsor-specific disposable cassettes that combine multiple unit operations—all focused on the core requirements of the final cell therapy product. Multiple automated cell engineering systems 400 can be integrated together into a large, multi-unit operation for production of large volumes of cells or multiple different cellular samples for individual patients.

Automated cell engineering system also further includes a user interface 420 for receiving input from a user. User interface 420 can be a touch pad, tablet, keyboard, computer terminal, or other suitable interface, that allows a user to input desired controls and criteria to the automated cell engineering system to control the automated processes and flow-path. Suitably, the user interface is coupled to a computer control system to provide instructions to the automated cell engineering system, and to control the overall activities of the automated cell engineering system. Such instructions can include when to open and close various valves, when to provide media or cell populations, when to increase or decrease a temperature, etc.

Automation of unit operations in cell therapy production provides the opportunity for universal benefits across allogeneic and autologous cell therapy applications. In the unique scenario of patient-specific, autologous cell products, and even more emphasized by the clinical success of these therapies, the advantages of automation are particularly compelling due to the significant micro-lot complexities of small batch GMP compliance, economics, patient traceability and early identification of process deviations. The associated emergence of complex manufacturing protocols draws attention to the fact that the value of end-to-end integration of automated unit operations in micro-lot cell production has not been a point of significant study. However, the expected demand for these therapies following their impending approval indicates that implementation of a fully closed end-to-end system can provide a much needed solution to manufacturing bottlenecks, such as hands-on-time and footprint.

FIG. 4A shows an exemplary set-up utilizing two combinatorial fluid switches (100 and 100′″″) along with two systems (300 and 300′), each of which include two combinatorial fluid switches. As indicated, each fluid switch has the capability of receiving up to four inputs, including one or more pass-through lines, as well as four outputs. As indicated, the inputs can include various connections to an input and output of cell culture chamber 402, inputs and outputs of various sampling ports, inputs and outputs of various media chambers, inputs and outputs of various filters, including magnetic filters, etc., as well as pump connections.

In further embodiments, provided here is a combinatorial fluid switch for controlling fluid flow through a plurality of fluid flow-paths, comprising: a plurality of fluid inputs; a first control valve, through which fluids from the plurality of fluid inputs are guided; a second control valve having individual fluid flow-paths, through which fluids from the plurality of fluid inputs are guided; and a plurality of fluid outputs, wherein the combinatorial fluid switch is configured to allow fluid flow through a designated combination of fluid inputs and fluid outputs. Various control valves can be utilized in the combinatorial switches, including those described herein and otherwise known in the art, allowing for the control of flow from multiple inputs, through multiple flow-paths, and out multiple outputs. The combinatorial nature of the fluid switches described herein allows for the tailored control of fluid flow, suitably within a automated biologic production system 400, and suitably within a cassette 410 of a cell engineering system. The combinatorial nature of the fluid switches also allows for the control of fluid flow in a closed system, maintaining the sterility and sealed nature of the fluid flow system.

Fluid flow-paths described herein suitably connect various temperature zones and can connect various elements of a cell engineering system, including cell separation, cell washing, cell isolation, etc. The flow-paths suitably direct various reagents, and include cell or virus transport flow-paths. The flow-paths can also be utilized to direct flow out of a cell engineering system (e.g., out of cassette) to external devices, such as electroporation or mechanoporation hardware, as well as microscope or optical elements (e.g., cameras), cell counting or cell sorting apparatus, etc.

The scale of the flow-paths can also be tailored by the use of varying tube diameter, including from mm to mm scale, as well the use of tubes to generate turbulent flow or increase shear, or tubes to eliminate or reduce turbulent flow or shear. Flow paths can also allow for mixing, or the combination of reagents, cells, virus, etc., and maintain their desired temperature and flow characteristics (i.e., steady flow or turbulent flow, as desired).

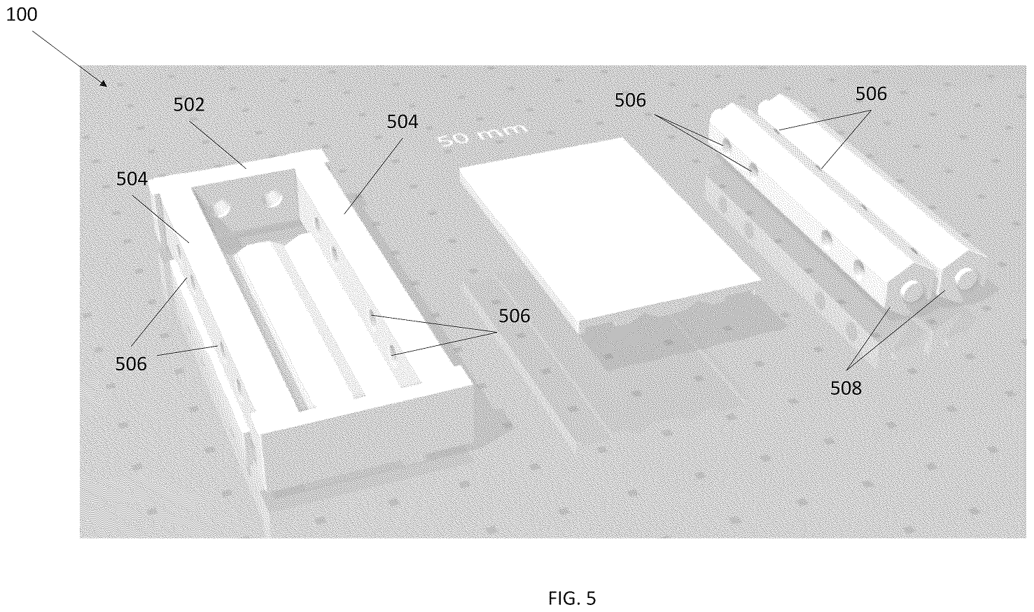

FIG. 5 shows an exemplary combinatorial fluid switch 100 as described herein, comprising: a housing 502 having two opposing sides 504, each side having four openings 506 passing therethrough; and two, two-position valves 508 disposed within the housing, each valve having four openings 506 passing therethrough. As described herein, the openings 506 in the sides 504 and the openings 506 in the two-position valves 508 are configured to receive tubing therethrough, so as to create four flow-paths within the combinatorial fluid switch, and the two-position valves are moveable within the housing to allow fluid flow through only one flow-path at a time.

In exemplary embodiments, the two-position valves are slidable within the housing. In further embodiments, the two-position valves are rotatable within the housing.

In suitably embodiments, the switch 100 shown in FIG. 5 further includes four tubing lines passing through the four openings in the sides and the four openings in the two-position valves.

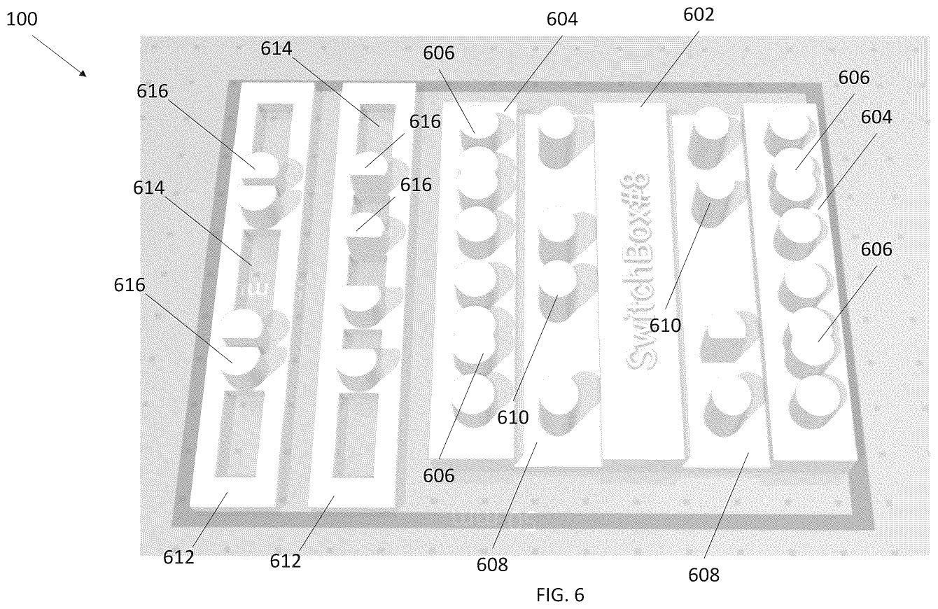

In further embodiments, FIG. 6 shows an combinatorial fluid switch 100, comprising: a support base 602 comprising two raised portions 604 and two recessed portions 608, the two raised portions 604 comprising a plurality of partitions 606 extending above the support base configured to allow tubing to pass therethrough so as to create four fluid flow-paths. The two recessed portions 608 each comprising four stationary compression members 610 extending above the support base. Switch 100 also includes two, two-position valves 612 having openings 614 to allow the stationary compression members 610 to pass through, the valves further comprising four movable compression members 616 configured to compress against a complementary, stationary compression member 610 on the support base 602 so as to constrict a tubing between the movable compression member 616 and the complementary, stationary 610 compression member. Suitably, the two-position valves are configured to slide along the support base and allow fluid flow through only one flow-path at a time.

In embodiments of the switch shown in FIG. 6, the two, two-position valves 612 are flat valves that slide within the two recessed portions 608 of the support base 602. The combinatorial fluid switch 100 also suitably further includes four tubing lines passing through the partitions, the stationary compression members and the movable compression members.

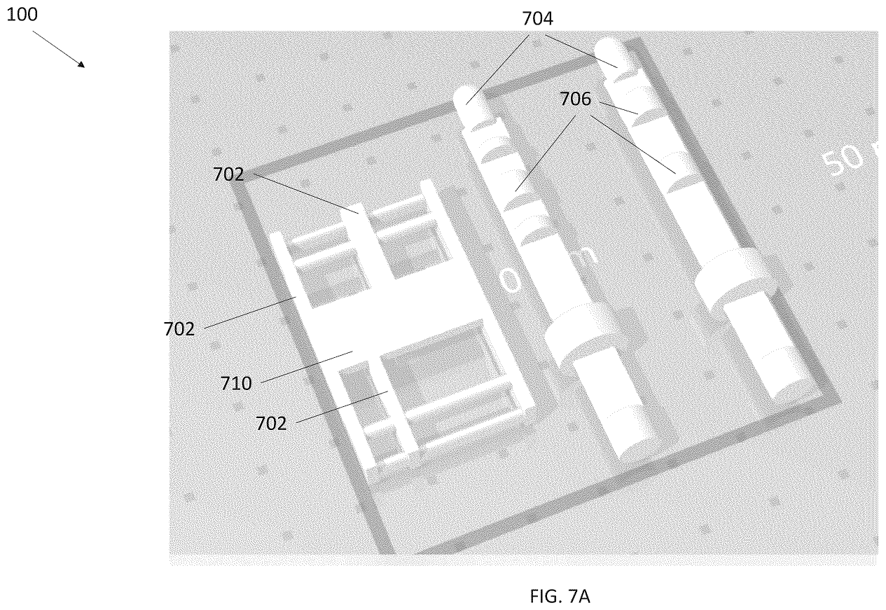

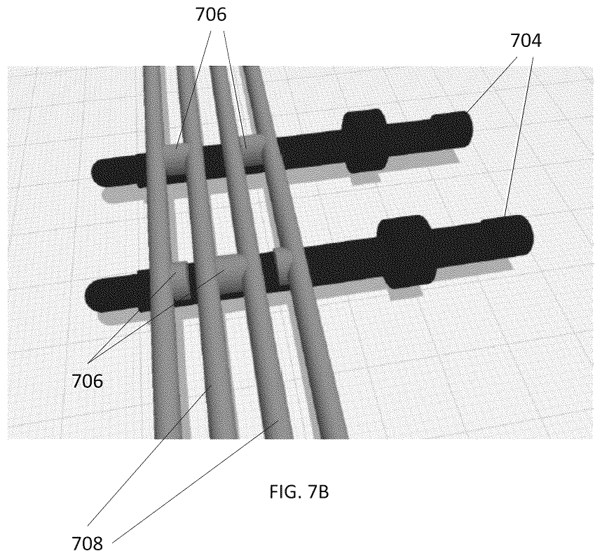

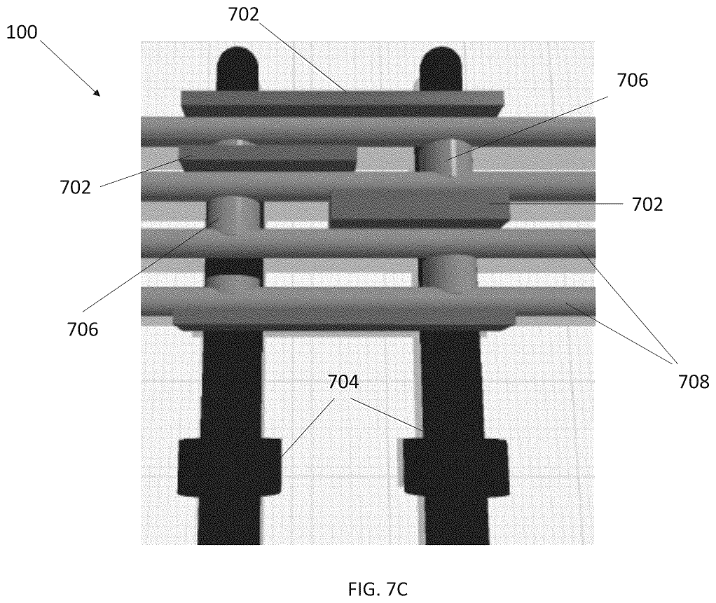

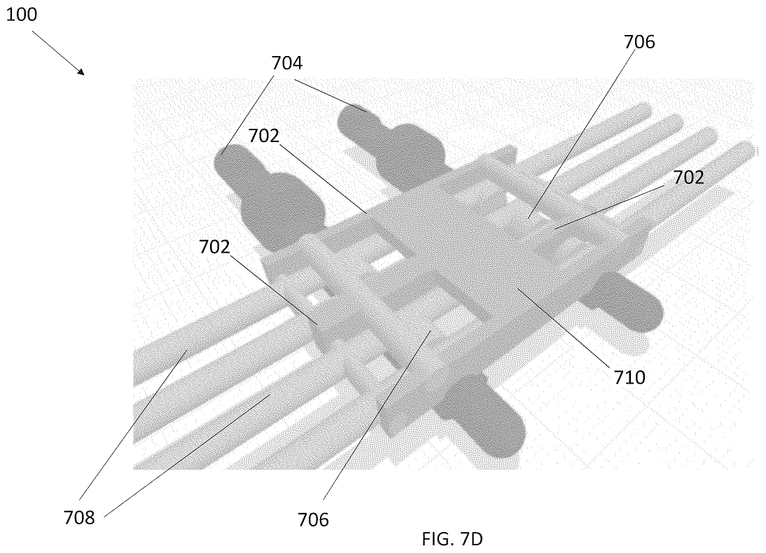

FIGS. 7A-7D show a further combinatorial fluid switch 100. In embodiments, the switch of FIGS. 7A-7D includes at least three, stationary compression members 702, a first two-position 704 valve having two movable compression members 706 integrated into the valve; and a second two-position valve 704 having three movable compression members 706 integrated into the valve. The movable compression members 706 of the first and second two-position valves 704 are configured to compress against a complementary, stationary compression member 702 so as to constrict a tubing 708 between the movable compression member and the complementary, stational compression member (see FIGS. 7B-7D). The two-position valves 704 are configured to slide and allow fluid flow through one flow-path at a time. In exemplary embodiments, the stationary compression modules 702 are disposed within a housing 710. Suitably, the two-position valves are trumpet valves. As shown in FIGS. 7B-7D, combinatorial fluid switch 100 of FIGS. 7A-7D can further four tubing lines 708 passing through the stationary compression members 702 and the movable compression members 706, to provide four fluid flow-paths. It should be noted that the moveable and stationary compression members shown in FIGS. 7A-7D can also be reversed, and still achieve the same control of the flow paths.

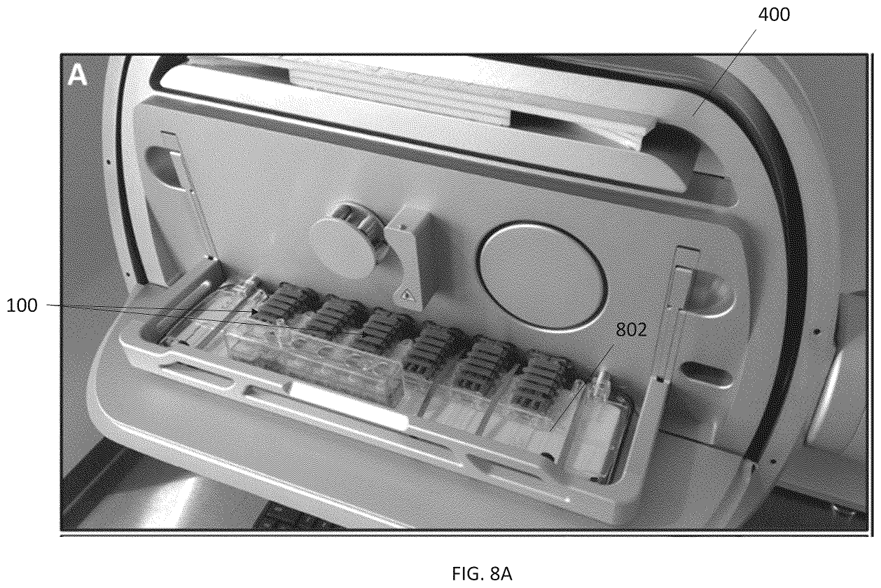

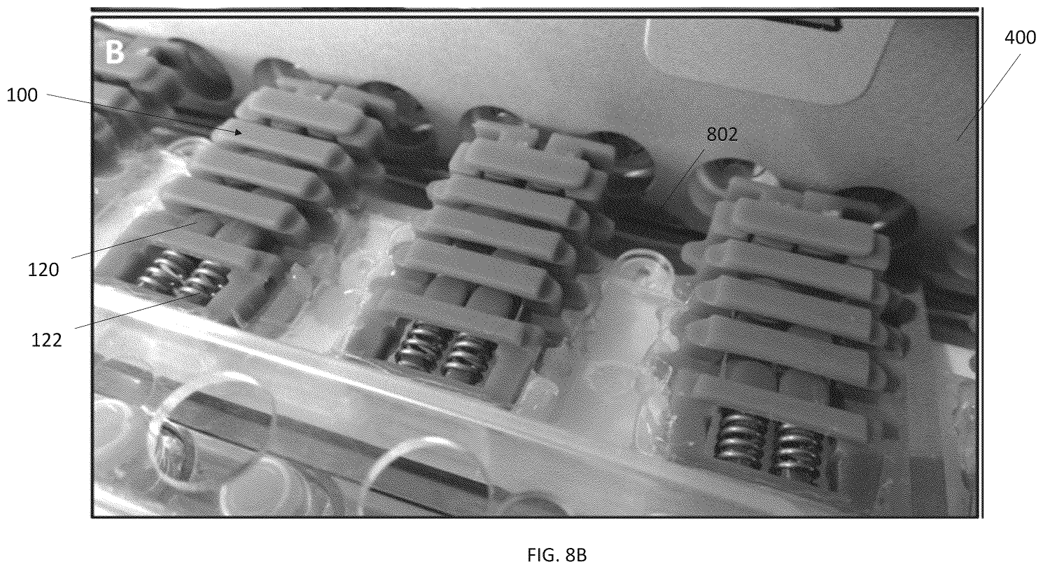

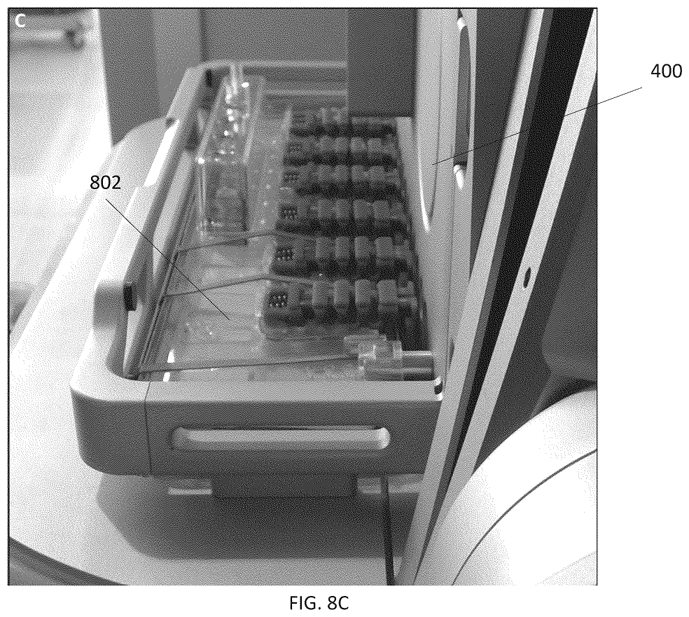

FIGS. 8A-8C show the integration of six (6) combinatorial fluid switches 100 for into an automated cell engineering system 400. As shown, each system 300 suitably includes two combinatorial switches 100. The systems are attached to a support manifold 802 of a cassette 410 (the rest of the cassette is removed for clarity). As described herein, the switches 100 suitably include 2, two position valves (120, 122), such as trumpet valves. The use of six (6) combinatorial fluid switches allows for control of 6×2² (i.e., 24) flow-paths through the automated cell engineering system 400. In further embodiments, the combinatorial switches can be integrated utilizing two combinatorial switches 100 at either end, with two systems 300 between them, resulting in 4+16+16+4=40 flow-path variations or combinations.

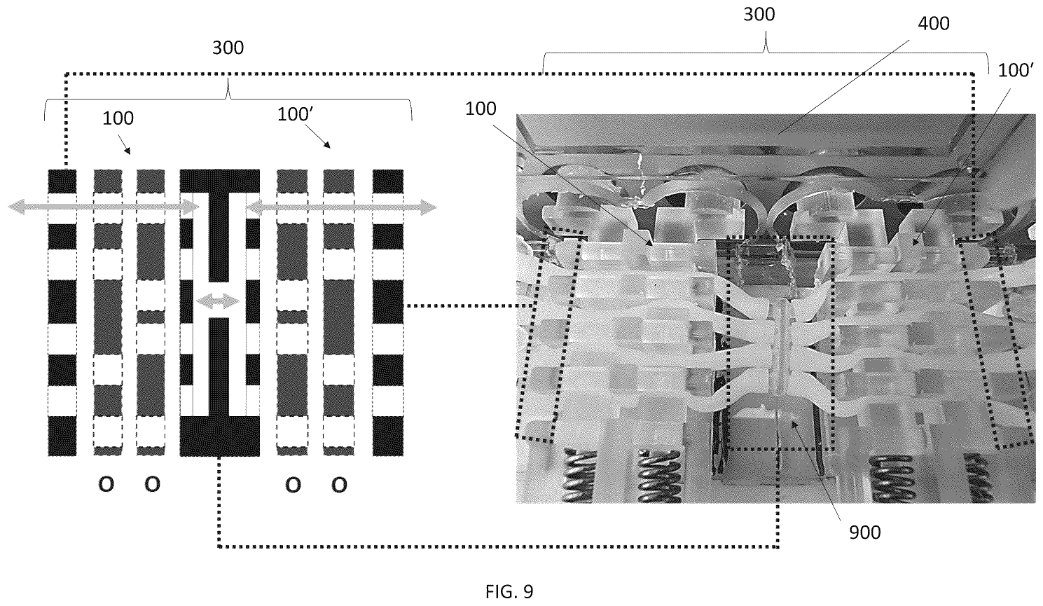

FIG. 9 shows the connection a system 300 containing two combinatorial switches 100 within a cassette 410, as described herein. Tubing connections 900 are also illustrated between the two combinatorial switches 100.

### Additional Exemplary Embodiments

Embodiment 1 is a combinatorial fluid switch for controlling fluid flow through a plurality of fluid flow-paths, comprising: a plurality of fluid inputs; a first two-position valve having individual fluid flow-paths, through which fluids from the plurality of fluid inputs are guided; a second two-position valve having individual fluid flow-paths, through which fluids from the plurality of fluid inputs are guided; and a plurality of fluid outputs, wherein the combinatorial fluid switch is configured to allow: fluid flow from a first fluid input to a first fluid output when the first two-position valve is in an open position and the second two-position valve is in an open position; fluid flow from a second fluid input to a second fluid output when the first two-position valve is in a closed position and the second two-position valve is in an open position; fluid flow from a third fluid input to a third fluid output when the first two-position valve is in an open position and the second two-position valve is in a closed position; and fluid flow from a fourth fluid input to a fourth fluid output when the first two-position valve is in a closed position and the second two-position valve is in a closed position.

Embodiment 2 includes the combinatorial fluid switch of embodiment 1, wherein the fluid inputs comprise tubing.

Embodiment 3 includes the combinatorial fluid switch of embodiment 1 or 2, wherein the fluid flow-paths comprise tubing.

Embodiment 4 includes the combinatorial fluid switch of any of embodiments 1-3, wherein the two-position valves are trumpet valves.

Embodiment 5 includes the combinatorial fluid switch of any of embodiments 1-4, wherein the fluid outputs comprise tubing.

Embodiment 6 includes the combinatorial fluid switch of any of embodiments 1-5, wherein the fluid inputs, the fluid flow-paths and the fluid outputs consist of at least four tubing lines.

Embodiment 7 includes the combinatorial fluid switch of any of embodiments 1-6, wherein each of the fluid inputs are fed from a single fluid source.

Embodiment 8 includes the combinatorial fluid switch of any of embodiments 1-7, wherein fluids within the fluid flow paths are not allowed to mix.

Embodiment 9 is a combinatorial fluid switch for controlling fluid flow through at least four fluid flow-paths, comprising: a first, a second, a third and a fourth fluid inputs; a first two-position valve having four fluid flow-paths, through which fluids from the first, second, third and fourth fluid inputs are guided; a second two-position valve having four fluid flow-paths, through which fluids from the first, second, third and fourth fluid inputs are guided; and a first, a second, a third and a fourth fluid outputs, wherein the combinatorial fluid switch is configured to allow: fluid flow from the first fluid input to the first fluid output when the first two-position valve is in an open position and the second two-position valve is in an open position; fluid flow from the second fluid input to the second fluid output when the first two-position valve is in a closed position and the second two-position valve is in an open position; fluid flow from the third fluid input to the third fluid output when the first two-position valve is in an open position and the second two-position valve is in a closed position; and fluid flow from the fourth fluid input to the fourth fluid output when the first two-position valve is in a closed position and the second two-position valve is in a closed position.

Embodiment 10 includes the combinatorial fluid switch of embodiment 9, wherein the fluid inputs comprise tubing.

Embodiment 11 includes combinatorial fluid switch of embodiment 9 or 10, wherein the fluid flow-paths comprise tubing.

Embodiment 12 includes the combinatorial fluid switch of any of embodiments 9-11, wherein the two-position valves are trumpet valves.

Embodiment 13 includes the combinatorial fluid switch of any of embodiments 9-12, wherein the fluid outputs comprise tubing.

Embodiment 14 includes the combinatorial fluid switch of any of embodiments 9-13, wherein the fluid inputs, the fluid flow-paths and the fluid outputs consist of four tubing lines.

Embodiment 15 includes the combinatorial fluid switch of any of embodiments 9-14, wherein each of the fluid inputs are fed from a single fluid source.

Embodiment 16 is a system for controlling fluid flow through at least sixteen fluid flow-paths, comprising: a first combinatorial fluid switch comprising: a first, a second, a third and a fourth fluid inputs; a first two-position valve having four fluid flow-paths, through which fluids from the first, second, third and fourth fluid inputs are guided; a second two-position valve having four fluid flow-paths, through which fluids from the first, second, third and fourth fluid inputs are guided; and a first, a second, a third and a fourth fluid outputs, wherein the first combinatorial fluid switch is configured to allow: fluid flow from the first fluid input to the first fluid output when the first two-position valve is in an open position and the second two-position valve is in an open position; fluid flow from the second fluid input to the second fluid output when the first two-position valve is in a closed position and the second two-position valve is in an open position; fluid flow from the third fluid input to the third fluid output when the first two-position valve is in an open position and the second two-position valve is in a closed position; and fluid flow from the fourth fluid input to the fourth fluid output when the first two-position valve is in a closed position and the second two-position valve is in a closed position; and a second combinatorial fluid switch fluidly connected to the first combinatorial fluid switch, the second combinatorial fluid switch comprising: a fifth, a sixth, a seventh and an eighth fluid inputs; a third two-position valve having four fluid flow-paths, through which fluids from the fifth, sixth, seventh and eighth fluid inputs are guided; a fourth two-position valve having four fluid flow-paths, through which fluids from the fifth, sixth, seventh and eighth fluid inputs are guided; and a fifth, a sixth, a seventh and an eighth fluid outputs, wherein the second combinatorial fluid switch configured to allow: fluid flow from the fifth fluid input to the fifth fluid output when the third two-position valve is in an open position and the fourth two-position valve is in an open position; fluid flow from the sixth fluid input to the sixth fluid output when the third two-position valve is in a closed position and the third two-position valve is in an open position; fluid flow from the seventh fluid input to the seventh fluid output when the third two-position valve is in an open position and the fourth two-position valve is in a closed position; and fluid flow from the eighth fluid input to the eighth fluid output when the third two-position valve is in a closed position and the fourth two-position valve is in a closed position.

Embodiment 17 is a system for controlling fluid flow through at least sixteen fluid flow-paths, comprising: a plurality of combinatorial fluid switches, each combinatorial fluid switch comprising: a first, a second, a third and a fourth fluid inputs; a first two-position valve having four fluid flow-paths, through which fluids from the first, second, third and fourth fluid inputs are guided; a second two-position valve having four fluid flow-paths, through which fluids from the first, second, third and fourth fluid inputs are guided; and a first, a second, a third and a fourth fluid outputs, wherein the first combinatorial fluid switch is configured to allow: fluid flow from the first fluid input to the first fluid output when the first two-position valve is in an open position and the second two-position valve is in an open position; fluid flow from the second fluid input to the second fluid output when the first two-position valve is in a closed position and the second two-position valve is in an open position; fluid flow from the third fluid input to the third fluid output when the first two-position valve is in an open position and the second two-position valve is in a closed position; and fluid flow from the fourth fluid input to the fourth fluid output when the first two-position valve is in a closed position and the second two-position valve is in a closed position.

Embodiment 18 includes the system of embodiment 16 or 17, wherein each of the combinatorial fluid switches is fluidly connected to each other.

Embodiment 19 includes the system of embodiment 16 or 17, comprising 4 or more combinatorial fluid switches.

Embodiment 20 includes the system of any one of embodiments 16-19, wherein the fluid inputs comprise tubing.

Embodiment 21 includes the system of any one of embodiments 16-20, wherein the fluid flow-paths comprise tubing.

Embodiment 22 includes the system of any one of embodiments 16-21, wherein each of the valves are trumpet valves.

Embodiment 23 includes the system of any one of embodiments 16-22, wherein the fluid outputs comprise tubing.

Embodiment 24 includes the system of any one of embodiments 16-23, wherein the fluid inputs, the fluid flow-paths and the fluid outputs consist of at least eight tubing lines.

Embodiment 25 is an automated biologic production system, comprising: an enclosable housing; a cassette contained within the enclosable housing, the cassette comprising: a cell culture chamber a combinatorial fluid switch of embodiment 1; a pumping system fluidly connected to the cell culture chamber and the combinatorial switch; one or more of a temperature sensor, a pH sensor, a glucose sensor, a lactose sensor, an oxygen sensor, a carbon dioxide sensor, and an optical density sensor; and mechanisms to automatically adjust one or more of a temperature, a pH level, a glucose level, a lactose level, an oxygen level, a carbon dioxide level, and an optical density.

Embodiment 26 is an automated biologic production system, comprising: an enclosable housing; a cassette contained within the enclosable housing, the cassette comprising: a cell culture chamber; a system of embodiment 8 or embodiment 9; a pumping system fluidly connected to the cell culture chamber and the system; one or more of a temperature sensor, a pH sensor, a glucose sensor, a lactose sensor, an oxygen sensor, a carbon dioxide sensor, and an optical density sensor; and mechanisms to automatically adjust one or more of a temperature, a pH level, a glucose level, a lactose level, an oxygen level, a carbon dioxide level, and an optical density.

Embodiment 27 includes the automated biologic production system of embodiment 25 or 26, configured to produce cells.

Embodiment 28 includes the automated biologic production system of any one of embodiments 25-27, further comprising a magnetic cell separation device.

Embodiment 29 includes the automated biologic production system of any one of embodiments 25-28, further comprising an electroporation device.

Embodiment 30 includes the automated biologic production system of any one of embodiments 25-29, wherein the automated biologic production system includes at least 16 fluid flow-paths.

Embodiment 31 includes the automated biologic production system of any one of embodiments 25-30, wherein the automated biologic production system includes at least 20 fluid flow-paths.

Embodiment 32 is a combinatorial fluid switch, comprising: a housing having two opposing sides, each side having four openings passing therethrough; and two, two-position valves disposed within the housing, each valve having four openings passing therethrough, wherein the openings in the sides and the openings in the two-position valves are configured to receive tubing therethrough, so as to create four flow-paths within the combinatorial fluid switch, and wherein the two-position valves are moveable within the housing to allow fluid flow through only one flow-path at a time.

Embodiment 33 includes the combinatorial fluid switch of embodiment 32, wherein the two-position valves are slidable within the housing.

Embodiment 34 includes the combinatorial fluid switch of embodiment 32, wherein the two-position valves are rotatable within the housing.

Embodiment 35 includes combinatorial fluid switch of any one of embodiments 32-34, further comprising four tubing lines passing through the four openings in the sides and the four openings in the two-position valves.

Embodiment 36 is a combinatorial fluid switch, comprising: a support base comprising two raised portions and two recessed portions, the two raised portions comprising a plurality of partitions extending above the support base configured to allow tubing to pass therethrough so as to create four fluid flow-paths, the two recessed portions each comprising four stationary compression members extending above the support base; two, two-position valves having openings to allow the stationary compression members to pass through, the valves further comprising four movable compression members configured to compress against a complementary, stationary compression member on the support base so as to constrict a tubing between the movable compression member and the complementary, stationary compression member, wherein the two-position valves are configured to slide along the support base and allow fluid flow through only one flow-path at a time.

Embodiment 37 includes the combinatorial fluid switch of embodiment 36, wherein the two, two-position valves are flat valves that slide within the two recessed portions of the support base.

Embodiment 38 includes the combinatorial fluid switch of embodiment 36 or 37, further comprising four tubing lines passing through the partitions, the stationary compression members and the movable compression members.

Embodiment 39 is a combinatorial fluid switch, comprising: at least three, stationary compression members; a first two-position valve having two movable compression members integrated into the valve; and a second two-position valve having three movable compression members integrated into the valve, wherein the movable compression members of the first and second two-position valves are configured to compress against a complementary, stationary compression member so as to constrict a tubing between the movable compression member and the complementary, stational compression member, wherein the two-position valves are configured to slide and allow fluid flow through one flow-path at a time.

Embodiment 40 includes the combinatorial fluid switch of embodiment 39, wherein the stationary compression modules are disposed within a housing.

Embodiment 41 includes the combinatorial fluid switch of embodiment 39 or 40, wherein the two-position valves are trumpet valves

Embodiment 42 includes the combinatorial fluid switch of any one of embodiments 39-41, further comprising four tubing lines passing through the stationary compression members and the movable compression members, to provide four fluid flow-paths.

Embodiment 43 is a combinatorial fluid switch for controlling fluid flow through a plurality of fluid flow-paths, comprising: a plurality of fluid inputs; a first control valve, through which fluids from the plurality of fluid inputs are guided; a second control valve having individual fluid flow-paths, through which fluids from the plurality of fluid inputs are guided; and a plurality of fluid outputs, wherein the combinatorial fluid switch is configured to allow fluid flow through a designated combination of fluid inputs and fluid outputs.

Embodiment 44 is a method for controlling fluid flow within a closed, cell engineering system, comprising: providing a plurality of fluid inputs; providing a plurality of fluid outputs; providing a plurality of flow-paths connecting the fluid inputs to the fluid outputs; providing a plurality of valves controlling the flow within the flow-paths, thereby controlling the direction, speed, duration and/or interval of fluid flow within the flow-paths.

It will be readily apparent to one of ordinary skill in the relevant arts that other suitable modifications and adaptations to the methods and applications described herein can be made without departing from the scope of any of the embodiments.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

All publications, patents and patent applications mentioned in this specification are herein incorporated by reference to the same extent as if each individual publication, patent or patent application was specifically and individually indicated to be incorporated by reference.

What is claimed is:

1. A combinatorial fluid switch for controlling fluid flow through a plurality of fluid flow-paths, comprising:
(a) a plurality of fluid inputs;
(b) a first two-position valve having individual fluid flow-paths, through which fluids from the plurality of fluid inputs are guided;
(c) a second two-position valve having individual fluid flow-paths, through which fluids from the plurality of fluid inputs are guided; and
(d) a plurality of fluid outputs,
wherein the combinatorial fluid switch is configured to allow:
fluid flow from a first fluid input to a first fluid output when the first two-position valve is in an open position and the second two-position valve is in an open position;
fluid flow from a second fluid input to a second fluid output when the first two-position valve is in a closed position and the second two-position valve is in an open position;
fluid flow from a third fluid input to a third fluid output when the first two-position valve is in an open position and the second two-position valve is in a closed position; and
fluid flow from a fourth fluid input to a fourth fluid output when the first two-position valve is in a closed position and the second two-position valve is in a closed position.

2. The combinatorial fluid switch of claim 1, wherein the fluid inputs comprise tubing.

3. The combinatorial fluid switch of claim 1 or claim 2, wherein the fluid flow-paths comprise tubing.

4. The combinatorial fluid switch of any one of claims 1-3, wherein the two-position valves are trumpet valves.

5. The combinatorial fluid switch of any one of claims 1-4, wherein the fluid outputs comprise tubing.

6. The combinatorial fluid switch of any one of claims 1-5, wherein the fluid inputs, the fluid flow-paths and the fluid outputs consist of at least four tubing lines.

7. The combinatorial fluid switch of any one of claims 1-6, wherein each of the fluid inputs are fed from a single fluid source.

8. The combinatorial fluid switch of any one of claims 1-7, wherein fluids within the fluid flow paths are not allowed to mix.

9. A combinatorial fluid switch for controlling fluid flow through at least four fluid flow-paths, comprising:
(a) a first, a second, a third and a fourth fluid inputs;
(b) a first two-position valve having four fluid flow-paths, through which fluids from the first, second, third and fourth fluid inputs are guided;
(c) a second two-position valve having four fluid flow-paths, through which fluids from the first, second, third and fourth fluid inputs are guided; and
(d) a first, a second, a third and a fourth fluid outputs, wherein the combinatorial fluid switch is configured to allow:
fluid flow from the first fluid input to the first fluid output when the first two-position valve is in an open position and the second two-position valve is in an open position;
fluid flow from the second fluid input to the second fluid output when the first two-position valve is in a closed position and the second two-position valve is in an open position;
fluid flow from the third fluid input to the third fluid output when the first two-position valve is in an open position and the second two-position valve is in a closed position; and
fluid flow from the fourth fluid input to the fourth fluid output when the first two-position valve is in a closed position and the second two-position valve is in a closed position.

10. The combinatorial fluid switch of claim 9, wherein the fluid inputs comprise tubing.

11. The combinatorial fluid switch of claim 9 or claim 10, wherein the fluid flow-paths comprise tubing.

12. The combinatorial fluid switch of any one of claims 9-11, wherein the two-position valves are trumpet valves.

13. The combinatorial fluid switch of any one of claims 9-12, wherein the fluid outputs comprise tubing.

14. The combinatorial fluid switch of any one of claims 9-13, wherein the fluid inputs, the fluid flow-paths and the fluid outputs consist of four tubing lines.

15. The combinatorial fluid switch of any one of claims 9-14, wherein each of the fluid inputs are fed from a single fluid source.

16. A system for controlling fluid flow through at least sixteen fluid flow-paths, comprising:
(a) a first combinatorial fluid switch comprising:
i. a first, a second, a third and a fourth fluid inputs;
ii. a first two-position valve having four fluid flow-paths, through which fluids from the first, second, third and fourth fluid inputs are guided;
iii. a second two-position valve having four fluid flow-paths, through which fluids from the first, second, third and fourth fluid inputs are guided; and
iv. a first, a second, a third and a fourth fluid outputs,
wherein the first combinatorial fluid switch is configured to allow:
fluid flow from the first fluid input to the first fluid output when the first two-position valve is in an open position and the second two-position valve is in an open position;
fluid flow from the second fluid input to the second fluid output when the first two-position valve is in a closed position and the second two-position valve is in an open position;
fluid flow from the third fluid input to the third fluid output when the first two-position valve is in an open position and the second two-position valve is in a closed position; and
fluid flow from the fourth fluid input to the fourth fluid output when the first two-position valve is in a closed position and the second two-position valve is in a closed position; and
(b) a second combinatorial fluid switch fluidly connected to the first combinatorial fluid switch, the second combinatorial fluid switch comprising:
i. a fifth, a sixth, a seventh and an eighth fluid inputs;
ii. a third two-position valve having four fluid flow-paths, through which fluids from the fifth, sixth, seventh and eighth fluid inputs are guided;
iii. a fourth two-position valve having four fluid flow-paths, through which fluids from the fifth, sixth, seventh and eighth fluid inputs are guided; and
iv. a fifth, a sixth, a seventh and an eighth fluid outputs,
wherein the second combinatorial fluid switch configured to allow:
fluid flow from the fifth fluid input to the fifth fluid output when the third two-position valve is in an open position and the fourth two-position valve is in an open position;
fluid flow from the sixth fluid input to the sixth fluid output when the third two-position valve is in a closed position and the third two-position valve is in an open position;
fluid flow from the seventh fluid input to the seventh fluid output when the third two-position valve is in an open position and the fourth two-position valve is in a closed position; and
fluid flow from the eighth fluid input to the eighth fluid output when the third two-position valve is in a closed position and the fourth two-position valve is in a closed position.

17. A system for controlling fluid flow through at least sixteen fluid flow-paths, comprising:
(a) a plurality of combinatorial fluid switches, each combinatorial fluid switch comprising:
i. a first, a second, a third and a fourth fluid inputs;
ii. a first two-position valve having four fluid flow-paths, through which fluids from the first, second, third and fourth fluid inputs are guided;
iii. a second two-position valve having four fluid flow-paths, through which fluids from the first, second, third and fourth fluid inputs are guided; and
iv. a first, a second, a third and a fourth fluid outputs,
wherein the first combinatorial fluid switch is configured to allow:
fluid flow from the first fluid input to the first fluid output when the first two-position valve is in an open position and the second two-position valve is in an open position;
fluid flow from the second fluid input to the second fluid output when the first two-position valve is in a closed position and the second two-position valve is in an open position;
fluid flow from the third fluid input to the third fluid output when the first two-position valve is in an open position and the second two-position valve is in a closed position; and
fluid flow from the fourth fluid input to the fourth fluid output when the first two-position valve is in a closed position and the second two-position valve is in a closed position.

18. The system of claim 16 or claim 17, wherein each of the combinatorial fluid switches is fluidly connected to each other.

19. The system of claim 16 or claim 17, comprising 4 or more combinatorial fluid switches.

20. The system of any one of claims 16-19, wherein the fluid inputs comprise tubing.

21. The system of any one of claims 16-20, wherein the fluid flow-paths comprise tubing.

22. The system of any one of claims 16-21, wherein each of the valves are trumpet valves.

23. The system of any one of claims 16-22, wherein the fluid outputs comprise tubing.

24. The system of any one of claims 16-23, wherein the fluid inputs, the fluid flow-paths and the fluid outputs consist of at least eight tubing lines.

25. An automated biologic production system, comprising:
(a) an enclosable housing;
(b) a cassette contained within the enclosable housing, the cassette comprising:
i. a cell culture chamber
ii. a combinatorial fluid switch of claim 1;
iii. a pumping system fluidly connected to the cell culture chamber and the combinatorial switch;
iv. one or more of a temperature sensor, a pH sensor, a glucose sensor, a lactose sensor, an oxygen sensor, a carbon dioxide sensor, and an optical density sensor; and
v. mechanisms to automatically adjust one or more of a temperature, a pH level, a glucose level, a lactose level, an oxygen level, a carbon dioxide level, and an optical density.

26. An automated biologic production system, comprising:
(a) an enclosable housing;
(b) a cassette contained within the enclosable housing, the cassette comprising:
i. a cell culture chamber;
ii. a system of claim 8 or claim 9;
iii. a pumping system fluidly connected to the cell culture chamber and the system;
iv. one or more of a temperature sensor, a pH sensor, a glucose sensor, a lactose sensor, an oxygen sensor, a carbon dioxide sensor, and an optical density sensor; and
v. mechanisms to automatically adjust one or more of a temperature, a pH level, a glucose level, a lactose level, an oxygen level, a carbon dioxide level, and an optical density.

27. The automated biologic production system of claim 25 or claim 26, configured to produce cells.

28. The automated biologic production system of any one of claims 25-27, further comprising a magnetic cell separation device.

29. The automated biologic production system of any one of claims 25-28, further comprising an electroporation device.

30. The automated biologic production system of any one of claims 25-29, wherein the automated biologic production system includes at least 16 fluid flow-paths.

31. The automated biologic production system of any one of claims 25-30, wherein the automated biologic production system includes at least 20 fluid flow-paths.

32. A combinatorial fluid switch, comprising:
(a) a housing having two opposing sides, each side having four openings passing therethrough; and
(b) two, two-position valves disposed within the housing, each valve having four openings passing therethrough,
wherein the openings in the sides and the openings in the two-position valves are configured to receive tubing therethrough, so as to create four flow-paths within the combinatorial fluid switch, and
wherein the two-position valves are moveable within the housing to allow fluid flow through only one flow-path at a time.

33. The combinatorial fluid switch of claim 32, wherein the two-position valves are slidable within the housing.

34. The combinatorial fluid switch of claim 32, wherein the two-position valves are rotatable within the housing.

35. The combinatorial fluid switch of any one of claims 32-34, further comprising four tubing lines passing through the four openings in the sides and the four openings in the two-position valves.

36. A combinatorial fluid switch, comprising:
(a) a support base comprising two raised portions and two recessed portions, the two raised portions comprising a plurality of partitions extending above the support base configured to allow tubing to pass therethrough so as to create four fluid flow-paths, the two recessed portions each comprising four stationary compression members extending above the support base;
(b) two, two-position valves having openings to allow the stationary compression members to pass through, the valves further comprising four movable compression members configured to compress against a complementary, stationary compression member on the support base so as to constrict a tubing between the movable compression member and the complementary, stationary compression member,
wherein the two-position valves are configured to slide along the support base and allow fluid flow through only one flow-path at a time.

37. The combinatorial fluid switch of claim 36, wherein the two, two-position valves are flat valves that slide within the two recessed portions of the support base.

38. The combinatorial fluid switch of claim 36 or claim 37, further comprising four tubing lines passing through the partitions, the stationary compression members and the movable compression members.

39. A combinatorial fluid switch, comprising:
(a) at least three, stationary compression members;
(b) a first two-position valve having two movable compression members integrated into the valve; and
(c) a second two-position valve having three movable compression members integrated into the valve,
wherein the movable compression members of the first and second two-position valves are configured to compress against a complementary, stationary compression member so as to constrict a tubing between the movable compression member and the complementary, stational compression member,
wherein the two-position valves are configured to slide and allow fluid flow through one flow-path at a time.

40. The combinatorial fluid switch of claim 39, wherein the stationary compression modules are disposed within a housing.

41. The combinatorial fluid switch of claim 39 or claim 40, wherein the two-position valves are trumpet valves

42. The combinatorial fluid switch of any one of claims 39-41, further comprising four tubing lines passing through the stationary compression members and the movable compression members, to provide four fluid flow-paths.

43. A combinatorial fluid switch for controlling fluid flow through a plurality of fluid flow-paths, comprising:
(a) a plurality of fluid inputs;
(b) a first control valve, through which fluids from the plurality of fluid inputs are guided;
(c) a second control valve having individual fluid flow-paths, through which fluids from the plurality of fluid inputs are guided; and
(d) a plurality of fluid outputs,
wherein the combinatorial fluid switch is configured to allow fluid flow through a designated combination of fluid inputs and fluid outputs.

44. A method for controlling fluid flow within a closed, cell engineering system, comprising:
(a) providing a plurality of fluid inputs;
(b) providing a plurality of fluid outputs;
(c) providing a plurality of flow-paths connecting the fluid inputs to the fluid outputs;
(d) providing a plurality of valves controlling the flow within the flow-paths, thereby controlling the direction, speed, duration and/or interval of fluid flow within the flow-paths.
